(19) United States
(12) Patent Application Publication
(10) Pub. No.: US 2018/0303076 A1
(43) Pub. Date: Oct. 25, 2018

(54) METHOD OF BREEDING DIPTERAN INSECTS

(71) Applicant: AGRIPROTEIN HOLDINGS LIMITED, Gibarltar (GI)

(72) Inventors: David DREW, Frankfurt Am Main (DE); Cameron RICHARDS, Cape Town (ZA); Cobus KOTZE, Kirstenhof (ZA); Jason DREW, Paarl (ZA); Roy RUDOLPHE, Constantia (ZA); Kieron DUNN, Kalk Bay (ZA)

(21) Appl. No.: 15/771,927

(22) PCT Filed: Oct. 28, 2016
(86) PCT No.: PCT/IB2016/056510

§ 371 Date: Apr. 27, 2018

(30) Foreign Application Priority Data
Oct. 28, 2015 (ZA) 2015/07993

(51) Int. Cl. A01K 67/033

(52) U.S. Cl. CPC A01K 67/033

(57) ABSTRACT

The invention provides a method of breeding the black soldier fly (Hermetia illucens), wherein the black soldier flies are exposed, at least some of the time, to artificial lighting that is blue in colour.

### FIELD OF INVENTION

The invention relates to apparatus and methods for breeding dipteran insects, particularly the black soldier fly (Hermetia illucens).

### BACKGROUND OF INVENTION

With a growing global population, the stresses on finding cheap and available resources to feed these people is becoming acute. As this large population increases in affluence so the dietary profiles move from grains and carbohydrates to proteins. Typical low cost farmed protein sources such as chicken and pork then place further supply stresses on global protein feeds. The impact on this has been seen on the price of fish meal and global fish stocks.

The ability to generate alternative protein sources is becoming imperative. Such alternatives can now be found in insect larvae meal which have similar protein and nutrition profiles to fish meal, and has been shown to be an excellent replacement for higher cost fish meal.

In addition to the application and use of the larval meal in feedstocks there are significant alternative applications of this technology including chitin extraction (CN 102585035 B, 2013, Preparation method for extracting chitosan from hermetia illucens), conversion of putrid wastes (U.S. Pat. No. 6,391,620 B1, P Olivier, 2002, Method for bio-conversion of putrescent wastes compost by products), and even biotechnology extractions for melanin (U.S. Pat. No. 8,815,539 B1, R Popa, 2014, Methods for producing melanin and inorganic fertilizer from fermentation leachates). Species evaluation has shown that the Black Soldier Fly (“BSF”) has been identified as one of the optimum breeds to utilize in an industrialised insect farming operation with advantages over many other alternatives including the House Fly (Musca Domestica). BSF are relatively indiscriminate feeders, robust and resistant to bacteria and pathogens, have reasonable harvestable size and rapid growth rates. Know-how currently exists to farm these insects at scale converting waste biomass into valuable animal feed.

The process of farming these insects is similar to that of chicken farming and involve two separate but co-dependent activities. Firstly the continuous rearing of a parent colony of “breeding” insects. These provide bulk quantities of eggs which are harvested for larval growth, the balance being fed back into the “breeding” area. Secondly the hatching and growth of the eggs into full size larvae that are harvested when optimal however prior to sixth instar or pre-pupation phase.

There are key obstacles that impact the efficiency of farming BSF at scale. Of primary importance is the need to maintain environmental conditions, including (but not exclusive to) constant temperature and humidity levels to ensure optimal breading and rearing (which are not the same). Setting up reliable and large scale climate controlled facilities is a key requirement to a predictable and reliable production process.

It is critically important that one ensure high levels of reproduction, fertility and egg laying. Any factors that inhibit this process will have a major impact on efficiency and output. Extensive research has been performed in this area including the impact of lighting on insect and BSF behavior.

The impact of lighting on insects is not a new area of research, and has been studied for many years. Artificial lighting is well established in poultry, hydroponics and other farming activities.

The effect of particular lighting conditions as part of the mating and laying (oviposition) processes has been studied to some extent, the most basic of these is obviously in sunlight (Tomberlin et al, 2002). However, given that insect rearing requires housing large colonies in an enclosed and therefore largely artificial environment, the need to find an artificial lighting solution is critical. Standard incandescent lighting has provided comparable results to that of sunlight.

The ability to enhance the mating and oviposition activity is crucial to improving harvest yield. Using artificial lighting to provide such enhancements is a key area of study. The only scientific account of using artificial lighting to enhance mating of BSF was using a 500 W quartz-iodine lamp, (Zhang et al. 2010).

To examine the impact of artificial lighting on the BSF mating activities one needs to examine all the elements that constitute adequate and enhancing illumination as well as how this impacts on the biological processes that give rise to mating and oviposition success and the enhancement thereof.

One has to consider:
The type of different lighting technologies to be used including incandescent, halogen (U.S. Pat. No. 8,733,284 B2, G Neil, 2012, Method and apparatus for breeding flying insects; where a quartz bromide lamp is used as part of a broader construction for growing BSD), high pressure sodium and other bulbs;
Lighting Wavelength (WO 2013166590 A1, J Aldana, 2013, A Contained systems to provide reproductive habitat for hermetia illucens; where a combination of 2 lamps was used in both the visual and non visible spectrum lighting);
The strength of the lighting or luminosity which is a factor of intensity and height above the cage;
The stability of the lighting to ensure consistent illumination and a balanced and homogeneous light environment (U.S. Pat. No. 4,556,010 A, B Persson, 1985, Method of controlling noxious insects; lighting intensity was varied to repress the insect's oviposition and mating instinct); and
Achieving an appropriate photo period and variations of the duration of lighting to both emulate natural conditions as well as enhance oviposition and mating).

For the purpose of this specification, in relation to the intensity of light at a particular frequency, the phrases “substantially exclusively” means without light having a relative intensity of more than 0.1 at any frequency outside the stated frequency range; and “substantially free of” has a corresponding meaning.

### SUMMARY OF THE INVENTION

According to the invention, there is provided a method of breeding BSF, wherein adult BSF are exposed, at least some of the time, to artificial lighting that is blue in colour.

The method may comprise the stimulation of at least one of mating and ovipositioning of BSF by exposing the adult BSF, at least some of the time, to artificial lighting that is blue in colour.

The artificial lighting may be substantially exclusively within the wavelength range having a lower limit of not less than 390 nm, more preferably 400 nm, and most preferably 410 nm, and an upper limit of not more than 520 nm, more preferably 500 nm, and most preferably 490 nm.

The artificial lighting may have a peak occurring at a wavelength between 455 nm and 480 nm, preferably between 460 nm and 472 nm, and most preferably at a wavelength of substantially about 466 nm.

The artificial lighting may have a dominant wavelength between 460 nm and 480 nm, more preferably between 468 nm and 746 nm, and most preferably substantially about 472 nm.

The artificial lighting may be provided by Light Emitting Diodes (“LED”s).

The methods of the invention may include an illumination cycle in a 24 hour period having at least one illuminated period, wherein the artificial lighting is switched on, and at least one dark period, wherein the artificial lighting is switched off. The illuminated period may be no longer than 12 hours, alternatively, no longer than 6 hours.

Alternatively, the illumination cycle in a period of 24 hours may include an intermittent light cycle comprising a plurality of intermittent illuminated periods interspersed with dark periods, followed by an extended dark period. The intermittent light period may comprise alternating illuminated periods and dark periods up to one hour each, and the extended dark period may be up to 6 hours.

### BRIEF DESCRIPTION OF THE DRAWINGS

The invention will now be described, by way of non-limiting example only, with reference to the following figures, wherein:

FIG. 1 is a representation of the visible spectrum, showing wave lengths of colours;

FIG. 2 is a graphic showing the mean number of mating pairs of BSF when exposed to red, green, or blue light, according to experiments by the applicant;

FIG. 3 is a diagram showing the spectrum of light emitted by a blue LED light source suitable for use in accordance with the invention; and

FIG. 4 is a graph comparing the mass of BSF eggs collected under blue light conditions compared with white light conditions, according to experiments conducted by the applicant.

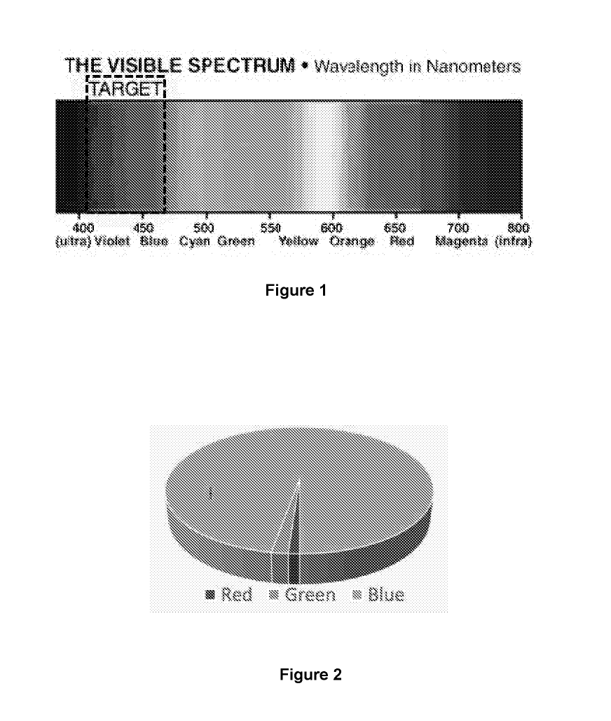

### DESCRIPTION OF SPECIFIC EMBODIMENTS

Various embodiments of the invention provide a method of using artificial lighting in the rearing and enhancement of oviposition and mating of the Black Soldier Fly (BSF).

The method involves the provision of artificial lighting in the visible blue lighting band.

Experiments by the applicant have demonstrated significant improvement in the level of BSF mating and oviposition under these conditions, as discussed further hereinbelow. Further, it is to be understood that improvements in the level of mating and oviposition are reasonably expected to result in increased yield of larvae.

### Experiment 1

Testing of this method was carried out using standard BSF rearing conditions. 12 kilograms of pupae were placed into a cage to eclose into ˜6,000 adults. The cages were held in an environment controlled room of ±28° C. with 80% relative humidity. Six cages were placed under three different coloured lights: red, green, and blue. Lumen was measured at a distance of 30 cm, to ensure that brightness was generally equivalent for each cage. The lights were on the same 12 hour light (06:00 h to 18:00 h) and dark (18:00 h to 06:00 h) schedule.

The number of mating pairs was recorded by two observers by sight over the course of 5 days (Jul. 7-Jul. 11, 2014). Two cages per colored light were counted each day at 12:00 h for a ten minute period. Pairs were considered mating once the pair was clasped in a straight line facing away from each other and had landed on a surface.

The red, green, and blue lights produced 0.2, 0.3, 17.3 mean mating pairs, respectively. Blue light resulted in 98% more BSF mating pairs observed when compared to red and green lights. A graphic showing these results is provided in FIG. 2.

### Experiment 2

A second experiment was conducted to compare the effect of blue light to white light.

The white light source used was a Dual Arc MH/HPS 1000w—HOR, having metal halide (MH) and high pressure sodium (HPS) bulbs, so as to provide a wide spectrum of lighting.

The mass of eggs retrieved daily was measured across an 18 day period, and the results are shown in FIG. 4, summarized as follows:

| | Ave daily | | | | |
|---|---|---|---|---|---|
| | egg (g) | Stdev | −95% conf | +95 conf | n |
| Blue Light | 0.784375 | 0.038054 | 0.709392 | 0.859358 | 120 |
| White Light | 0.151319 | 0.032956 | 0.086382 | 0.216257 | 144 |

### Further Discussion

Surprisingly, in light of the Zhang and Aldana references cited above, tests conducted by the Applicant have established that significant improvement in the level of BSF mating and oviposition occurs with the provision of artificial lighting in the visible, generally blue, lighting band. It is therefore understood that the exposure of adult BSF to artificial lighting in the visible, generally blue, lighting band (however the blue character is defined, as discussed further hereinbelow) is expected to have the effect of stimulating mating and/or ovipositioning.

Further, it is to be expected that improvements in the level of mating and oviposition are reasonably expected to result in increased numbers of fertilized eggs, and ultimately increased yield of larvae. This is indeed the experience of the Applicant.

Further testing determined a target wavelength specifications encompass the following range of 410 nm and 490 nm, with a dominant wavelength at 472 nm and the peak occurring at 466 nm.

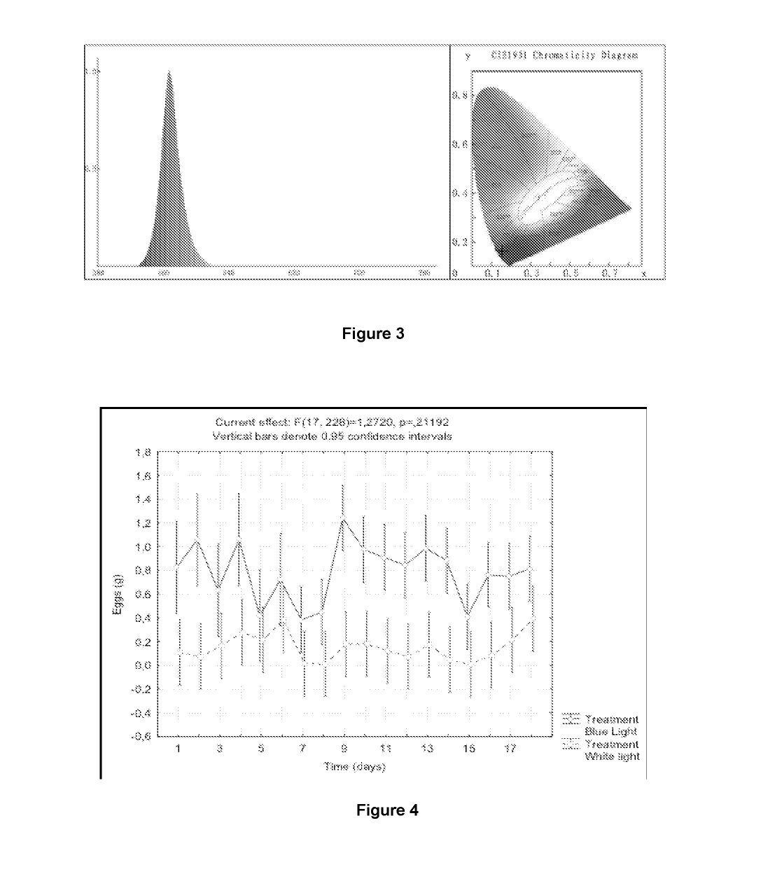

The method involves the use and application of Light Emitting Diodes (LED) of a specific range of wavelength to provide an enhanced environment that stimulates oviposition and mating of BSF. These LEDs are capable of emitting lighting between 410 nm and 490 nm with the targeted colour profile outlined in FIG. 3. The LED lights used by the Applicant have a dominant wavelength of about 472 nm, and a peak wavelength of about 466 nm.

The Applicant expects that the beneficial effects of the blue artificial lighting will be experienced across a broader range of lighting having a generally blue character as follows:
The artificial lighting may be substantially exclusively within the wavelength range having a lower limit of not less than 390 nm, more preferably 400 nm, and most preferably 410 nm, and an upper limit of not more than 520 nm, more preferably 500 nm, and most preferably 490 nm;
The artificial lighting may have a peak occurring at a wavelength between 455 nm and 480 nm, preferably between 460 nm and 472 nm, and most preferably at a wavelength of substantially about 466 nm; and/or
The artificial lighting may have a dominant wavelength between 460 nm and 480 nm, more preferably between 468 nm and 746 nm, and most preferably substantially about 472 nm.

In this regard, it should be understood that the various necessary and optional features of the invention as described herein would be applicable to the methods of the invention, irrespective of the manner in which the blue character of the artificial light is defined.

Additional value in utilising LEDs was identified in the substantial energy savings provided by these lights, their capacity for low flicker/oscillation operation (especially when using DC current), their robustness, their ability to tolerate high temperature and high humidity environments typical of the fly rearing cages, and their long lifespans requiring limited replacement. In addition LEDs generate far lower levels of heat than traditional lighting thereby reducing the load on HVAC systems and variance in environment within the cage.

The method allows for altered photoperiods that allow for shorter than 12 hour illumination. Such periods may be as low as one hour illuminated and one hour with no lighting for a period of 6 hours, thereby halving the electrical costs to light the environment. Reduced photo periods have shown marginal impact on mating and oviposition and in some cases have been shown to promote oviposition.

The applicant expects that the positive effects shown in the abovementioned experiments may be experienced without the total exclusion of light outside the spectrum used in these experiments. Accordingly, applicant envisages that the method of the invention may use artificial lighting within the wavelength range, peak range and dominant wavelength range set out hereinabove.

It will be appreciated that various embodiments of the invention may be made without departing from the scope and spirit of the invention as set out in the accompanying claims.

1. A method of breeding BSF, wherein adult BSF are, at least some of the time, exposed to artificial lighting, characterised in that the artificial lighting is blue.

2. A method as claimed in claim 1, wherein the blue lighting is characterised in that the lighting is substantially exclusively within the wavelength range having a lower limit of not less than 390 nm and an upper limit of not more than 520 nm.

3. A method as claimed in claim 2 wherein the lower limit is not less than 400 nm.

4. A method as claimed in claim 2, wherein the lower limit is not less than 410 nm.

5. A method as claimed in claim 2 wherein the upper limit is not more than 500 nm.

6. A method as claimed in claim 2 wherein the upper limit is not more than 490 nm.

7. A method as claimed in claim 1, wherein the blue lighting is characterised in that the lighting has a dominant wavelength at a wavelength between 460 nm and 480 nm.

8. A method as claimed in claim 7, wherein the dominant wavelength is between 468 nm and 476 nm.

9. A method as claimed in claim 7, wherein the dominant wavelength is substantially about 472 nm.

10. A method as claimed in claim 7, wherein the artificial lighting is substantially free of light having a wavelength below 390 nm.

11. A method as claimed in claim 1, wherein the blue lighting is characterised in that the lighting has a peak occurring at a wavelength between 455 nm and 480 nm.

12. A method as claimed in claim 11, wherein the peak occurs at a wavelength between 460 nm and 472 nm

13. A method as claimed in claim 11, wherein the peak occurs at a wavelength of substantially about 466 nm.

14. A method as claimed in claim 1, wherein the artificial lighting is provided by Light Emitting Diodes (“LED”s).

15. A method as claimed in claim 1, wherein the adult BSF are exposed to the blue artificial lighting to stimulate at least one of mating and ovipositioning.

16. A method as claimed in claim 1, including an illumination cycle in a 24 hour period, having at least one illuminated period, wherein the artificial lighting is switched on, and at least one dark period, wherein the artificial lighting is switched off.

17. A method as claimed in claim 16, wherein the illuminated period is no longer than 12 hours.

18. A method as claimed in claim 16, wherein the illuminated period is no longer than 6 hours.

19. A method as claimed in claim 16, including an intermittent light cycle comprising a plurality of intermittent illuminated periods interspersed with dark periods, followed by an extended dark period.

20. A method as claimed in claim 19, wherein the intermittent light period comprises alternating illuminated periods and dark periods up to one hour each, and the extended dark period is up to 6 hours.
